(19) United States
(12) Patent Application Publication
(10) Pub. No.: US 2018/0055346 A1
(43) Pub. Date: Mar. 1, 2018

(54) LIGHT CONTROL DEVICE AND DIAGNOSTIC DEVICE

(71) Applicant: OLYMPUS CORPORATION, Tokyo (JP)

(72) Inventor: Tatsuhiko OKITA, Akiruno-shi (JP)

(73) Assignee: OLYMPUS CORPORATION, Tokyo (JP)

(21) Appl. No.: 15/788,080

(22) Filed: Oct. 19, 2017

Related U.S. Application Data
(63) Continuation of application No. PCT/JP2015/061952, filed on Apr. 20, 2015.

(51) Int. Cl. A61B 1/00; H04N 5/225; G02B 23/24

(52) U.S. Cl. CPC A61B 1/00186; H04N 2005/2255; G02B 23/2461; H04N 5/2256

(57) ABSTRACT

A light control device that performs, on an optical path of a light, optical control with respect to the light, comprises a blade, a rotary shaft member, a light control element, an upper substrate, and a lower substrate. The upper substrate is provided perpendicular to the rotary shaft member on one end of the rotary shaft member, has a first side surface positioned on the optical path side, and is formed to define a first space lateral to the first side surface and above a blade. The lower substrate is provided perpendicular to the rotary shaft member on other end of the rotary shaft member, has a second side surface positioned on the optical path side, and is formed to define a second space lateral to the second side surface and below the blade.

### CROSS-REFERENCE TO RELATED APPLICATION

This application is a Continuation Application of PCT Application No. PCT/JP2015/061952, filed Apr. 20, 2015, the entire contents of which are incorporated herein by reference.

### BACKGROUND OF THE INVENTION

### 1. Field of the Invention

The present invention relates generally to a light control device that performs optical control of an optical path by placing and removing a light control element on the optical path, and a medical or an industrial diagnostic device using said device.

### 2. Description of the Related Art

There is a light control device that performs optical control of an optical path by arranging a light control element, such as a shutter, a lens, or a filter, on the optical path, such as a photographing optical path. Such light control device has been used for medical equipment or industrial equipment, such as an endoscope in the medical field or the industrial field, a treatment tool, or an auxiliary tool. Among such equipment, an endoscope, for example, is inserted into a cavity of a subject of a human body, etc. to perform diagnosis or treatment, etc. Therefore, a light control device used for such equipment is required to be miniaturized. Even industrialized endoscopes are required to be miniaturized for inspecting details of machinery, etc.

For example, U.S. Pat. No. 9,164,355, U.S. Pat. No. 9,164,356 and Jpn. Pat. Appln. KOKAI Publication No. 9-22042 suggest configurations for achieving such miniaturization.

### BRIEF SUMMARY OF THE INVENTION

According to a first aspect of the present invention, there is provided a light control device that performs, on an optical path of a light to be received by an imaging element, optical control with respect to the light, the light control device comprising: a blade that has a distal end and a proximal end, and rotationally moves about the proximal end in a direction perpendicular to the optical path so as to be placed on and removed from the optical path; a rotary shaft member that has one end and another end, the rotary shaft member being provided on the proximal end of the blade in a manner that perpendicularly penetrates the blade, and rotating about a rotation axis that passes through the one end and the other end, to rotationally move the blade; a light control element that is provided on the blade, and, when positioned on the optical path by the rotational movement of the blade, performs optical control with respect to the light; an upper substrate that is provided perpendicular to the rotary shaft member on the one end of the rotary shaft member, the upper substrate having a first upper surface, a first lower surface, and a first side surface positioned on the optical path side, and being formed to define a first space lateral to the first side surface and above the blade; and a lower substrate that is provided perpendicular to the rotary shaft member on the other end of the rotary shaft member, the lower substrate having a second upper surface, a second lower surface, and a second side surface positioned on the optical path side, and being formed to define a second space lateral to the second side surface and below the blade.

According to a second aspect of the present invention, there is provided a diagnostic device, comprising the light control device according to the first aspect of the present invention, wherein the light control device performs optical control with respect to the light.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWING

The accompanying drawings, which are incorporated in and constitute apart of the specification, illustrate embodiments of the invention, and together with the general description given above and the detailed description of the embodiments given below, serve to explain the principles of the invention.

FIG. 1 is an exploded configuration diagram of a light control device according to a first embodiment of the present invention.

FIG. 2 is an assembly diagram showing the device.

FIG. 3 is a circuit configuration diagram showing an electric power supply system of an electromagnetic drive source in the device.

FIG. 4A is a side view of a conventional light control device.

FIG. 4B is a side view of the light control device according to the first embodiment.

FIG. 5 is a perspective view showing an insertion section of an endoscope.

FIG. 6 is an exploded configuration diagram of a light control device according to a second embodiment of the present invention.

FIG. 7 is an assembly diagram showing the device.

FIG. 8A is a perspective view showing a state in which one of a plurality of optical paths is selected in the device.

FIG. 8B is a perspective view showing a state in which another optical path is selected in the device.

FIG. 9 is a diagram showing each space that is formed since a lower substrate and an upper substrate are not present in the device.

FIG. 10 is a diagram showing various units of a diagnostic device arranged in a space where the lower substrate and the upper substrate are not present in the device.

### DETAILED DESCRIPTION OF THE INVENTION

### First Embodiment

In the following, a light control device according to a first embodiment of the present invention will be explained with reference to the drawings.

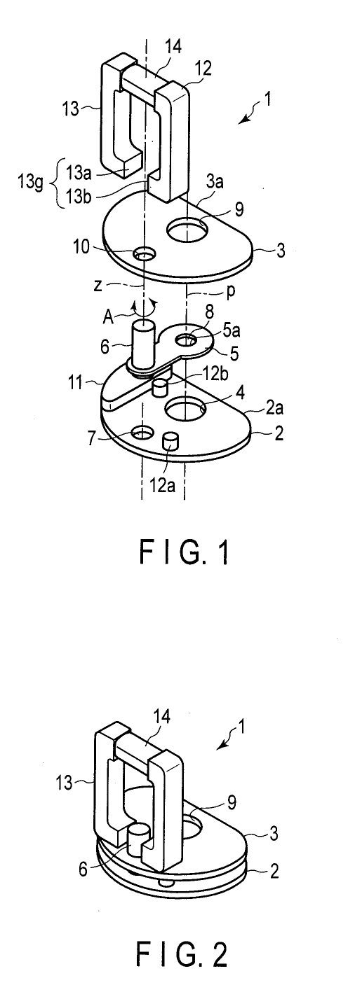

FIG. 1 shows an exploded configuration diagram of the light control device, and FIG. 2 shows an assembly diagram of the device. This light control device (hereinafter, referred to as the present device) 1 includes a lower substrate 2 and an upper substrate 3. The lower substrate 2 and the upper substrate 3 each have a circular shape, and a notch 2a or 2b formed on a part thereon.

On the lower substrate 2 is provided an optical path hole 4 for passing an optical path p through, and a shaft bearing hole 7 for bearing a rotary shaft member 6. The rotary shaft member 6 is provided with a blade 5. The rotary shaft member 6 and the blade 5 form a rotary member.

If the present device 1 is to be provided in, for example, an endoscope which is a medical diagnostic device, the present device 1 will be incorporated in the endoscope so that the optical path p coincides with an optical axis of a photographing system provided at a distal end of an endoscope insertion section. This photographing system includes, for example, an imaging element for imaging an image inside a subject when being inserted inside the body of a subject such as a human body, etc., and an illumination system for illuminating the inside of the subject. Accordingly, the present device 1 will be incorporated in the endoscope in a manner that the optical path p passes through the imaging element of the photographing system.

The rotary shaft member 6 is formed cylindrically, and is magnetized. This rotary shaft member 6, for example, is divided in two at a plane passing through a central axis of the cylindrical rotary shaft member 6 to form two semicircular cylinders, one of which is magnetized by an N-pole, and the other of which is magnetized by an S-pole.

The rotary shaft member 6 rotates inside the shaft bearing hole 7 in an arrow A direction with an axial direction z of the rotary shaft member 6 as the center of rotation. The axial direction z of the rotary shaft member 6 is in parallel with the optical path p. On the rotary shaft member 6, the blade 5 is provided in a direction perpendicular to the axial direction z of the rotary shaft member 6. One end of the blade 5 is provided on the rotary shaft member 6 in the manner mentioned above, and the other end is provided with a hole 5a for attaching a light control element 8 or to function as a light control element. This light control element 8 is, for example, a shutter, a lens, a shielding plate, or a filter. Accordingly, by rotating the rotary shaft member 6, the light control element 8 is rotationally moved around the rotary shaft member 6 via the blade 5. The plane on which the blade 5 rotationally moves is within a plane perpendicular to the axial direction z of the rotary shaft member 6.

In the same manner as the lower substrate 2, the upper substrate 3 is provided with an optical path hole 9 for passing the optical path p through, and a shaft bearing hole 10 for bearing the rotary shaft member 6 for rotating the blade 5.

The lower substrate 2 is provided with a spacer 11 and two stoppers 12a and 12b. The upper substrate 3 is assembled to the lower substrate 2 by being fixed to the spacer 11 and the stoppers 12a and 12b by adhesion, etc. Here, the lower substrate 2 and the upper substrate 3 are assembled so that the optical path hole 4 of the lower substrate 2 and the optical path hole 9 of the upper substrate 3 are arranged on the optical path p, and the shaft bearing hole 7 of the lower substrate 2 and the shaft bearing hole 10 of the upper substrate 3 are arranged in the axial direction z of the rotary shaft member 6.

The spacer 11 defines the interval between the lower substrate 2 and the upper substrate 3.

Each of the stoppers 12a and 12b defines a position at which the rotation of the blade 5 should be stopped when the blade 5 is rotated about the rotary shaft member 6. The stopper 12a, for example, stops the hole 5a of the blade 5 at a position away from the optical path holes 4 and 9, that is, a position away from the optical path p. The stopper 12b, for example, stops the hole 5a of the blade 5 at a position that matches the optical path holes 4 and 9, that is, a position on the optical path p. By stopping the blade 5 by the stopper 12b, for example, the light control element 8 such as the shutter, the lens, the shielding plate, or the filter is arranged on the optical path p. The light control element 8 is optically controlled for the optical path p in this manner.

When manufacturing the lower substrate 2 and the upper substrate 3, instead of assembling the upper substrate 3 to the lower substrate 2 by providing the spacer 11 and the stoppers 12a and 12b on the lower substrate 2 in the above manner, the opposite may be performed. That is, the spacer 11 and the stoppers 12a and 12b may be provided on the upper substrate 3, to which the lower substrate 2 will be assembled. Alternatively, a spacer may be provided to one of the upper substrate 3 or the lower substrate 2, and the stoppers 12a and 12b may be provided on the other substrate. The one member maybe used as the spacer and the stopper(s).

An electromagnetic drive source 12 for rotating the rotary shaft member 6 is provided in a tilted manner against the upper surface of the upper substrate 3. The electromagnetic drive source 12 rotationally moves the blade 5 by rotating the rotary shaft member 6, and causes the light control element 8 provided on the blade 5 to rotationally move around the rotary shaft member 6. The matter of the electromagnetic drive source 12 being provided in a tilted manner will be explained later on. The electromagnetic drive source 12 generates an electromagnetic force to rotate the rotary shaft member 6. The electromagnetic drive source 12 comprises, for example, a magnetic member 13 that is rectangular with a gap 13g formed thereon, and a coil 14 that is formed by being wound around the magnetic member 13.

The magnetic member 13 is, for example, formed rectangular with four sides. On one of the four sides, the gap 13g is formed. The gap 13g has gap ends 13a and 13b that face each other. These gap ends 13a and 13b form an opening on the rectangular magnetic member 13. The rotary shaft member 6 magnetized by the N-pole and S-pole in the manner mentioned above is arranged between these gap ends 13a and 13b.

In the case where this magnetic member 13 is installed on the upper surface of the upper substrate 3, the side on which the gap 13g is formed is installed in contact with the upper surface of the upper substrate 3.

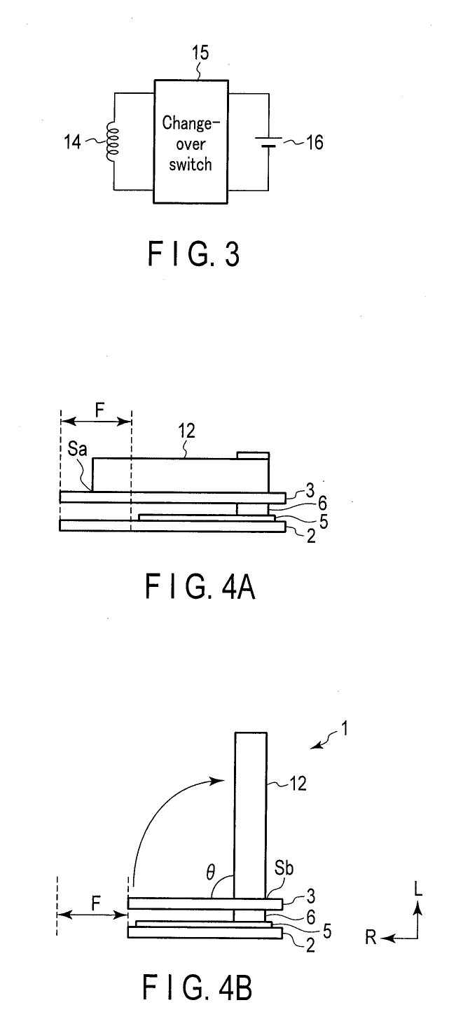

On the side of the magnetic member 13 facing the side on which the gap 13g is formed, the coil 14 is provided. As shown, for example, in FIG. 3, the coil 14 is connected to a direct-current power source 16 via a change-over switch 15. The change-over switch 15 performs a first switchover which, while adding a positive electrode of the direct-current power source 16 to one end of the coil 14, adds a negative electrode of the direct-current power source 16 to the other end, and a second switchover which, while adding the negative electrode of the direct-current power source 16 to one end of the coil 14, adds the positive electrode of the direct-current power source 16 to the other end. This change-over switch 15 may perform the first and the second switchovers manually, or by receiving switchover instructions from, for example, an assist unit that assists insertion/removal of an endoscope.

In the above power supply system, when the positive electrode is applied from the direct-current power source 16 to one end of the coil 14 via the change-over switch 15, and the negative electrode is applied to the other end, a magnetic field is generated by electromagnetic induction of the coil 14. A magnetic flux of this magnetic field passes through the magnetic member 13 and between the gap ends 13a and 13b of the magnetic member 13. The flow of this magnetic flux causes a magnetic circuit to be formed in the magnetic member 13. At this state, since the rotary shaft member 6 is provided between the gap ends 13a and 13b of the magnetic member 13, the magnetic flux is applied to the rotary shaft member 6. The optical path p is not arranged in this magnetic circuit; however, is arranged outside the magnetic circuit.

Since the rotary shaft member 6 is magnetized by the N-pole and the S-pole, suction power and repulsive power are generated by the magnetic flux between the gap ends 13a and 13b and the N-pole and the S-pole of the rotary shaft member 6, thereby rotating the rotary shaft member 6 in the arrow A direction. The direction in which the rotary shaft member 6 rotates depends on the direction in which the magnetic flux faces. When the rotary shaft member 6 is rotated, the blade 5 provided on the rotary shaft member 6 rotationally moves around the rotary shaft member 6. The blade 5 rotationally moves until it is stopped by abutting the stopper 12a or 12b. For example, when the blade 5 abuts the stopper 12a, the light control element 8 provided on the blade 5 stops at a position away from the optical path holes 4 and 9, that is, away from the optical path p. On the other hand, when the blade 5 abuts the stopper 12b, the light control element 8 provided on the blade 5 stops at a position matching the optical path holes 4 and 9, that is, on the optical path p.

As mentioned above, the electromagnetic drive source 12 is provided in a tilted manner against the upper surface of the upper substrate 3. Specifically, the magnetic member 13 on which the coil 14 is provided is arranged in a tilted manner against the upper surface of the upper substrate 3. In this manner, the installation area of the electromagnetic drive source 12 on the upper surface of the upper substrate 3 can be made smaller compared to the case in which the electromagnetic drive source 12 is not tilted. In other words, FIG. 4A shows a case of a conventional light control device in which the electromagnetic drive source 12 is not tilted, and FIG. 4B shows a case of the present device 1 in which the electromagnetic drive source 12 is tilted.

If the electromagnetic drive source 12 is provided perpendicular to the axial direction z of the rotary shaft member 6, that is, in parallel with the upper surface of the upper substrate 3 as shown in FIG. 4A, the electromagnetic drive source 12 has an installation area Sa, in which the electromagnetic drive source 12 is projected on the upper surface of the upper substrate 3. On the other hand, in the present embodiment, the electromagnetic drive source 12 is provided in a tilted manner against the upper surface of the upper substrate 3 by an angle θ as shown in FIG. 4B, and has an installation area Sb, in which the electromagnetic drive source 12 is projected on the upper surface of the upper substrate 3. The installation area Sb is smaller than the installation area Sa.

The installation area Sb of the electromagnetic drive source 12 becomes smaller as the angle θ of the tilt against the upper surface of the upper substrate 3 increases. As shown in FIG. 4A, the tilted angle θ of when the electromagnetic drive source 12 is provided in parallel with the upper substrate 3, is 0°.

A tilt θ of the electromagnetic drive source 12 is set within the range of 0<θ<180°, unless the optical path p is not shielded. More preferably, the tilt θ of the electromagnetic drive source 12 is set within the range of 0<θ≦90°. Furthermore, it is most favorable to set the tilt θ of the electromagnetic drive source 12 to 90°. FIG. 4B shows a case in which the tilted angle θ of the electromagnetic drive source 12 is 90°, which is the most favorable.

According to the first embodiment comprising the above structure, since the electromagnetic drive source 12 is provided in a tilted manner against the upper surface of the upper substrate 3 by angle θ, compared to the case in which the electromagnetic drive source 12 is not tilted, the installation area Sb of the electromagnetic drive source 12 with respect to the upper surface of the upper substrate 3 can be made smaller. Therefore, as shown in FIGS. 4A and 4B, the size of the upper substrate 3 (and the lower substrate 2) can be made smaller by, for example, dimension F in the case of tilting the electromagnetic drive source 12 than in the case of not tilting the electromagnetic drive source 12. If the present device 1 is incorporated in a medical or an industrial diagnostic device, such as an endoscope, the direction of the dimension F will be a radial direction of an insertion section distal end of the endoscope.

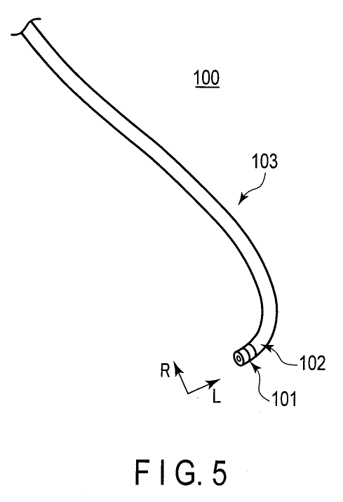

As shown in FIG. 5, for example, the insertion section 100 of the endoscope has a hard distal end 101 arranged at its distal end, and comprises on the proximal end side of the hard distal end 101 an active bending portion 102 that bends in accordance with the operator's operation, and a passive bending portion 103 that bends along the inner surface shape of the object into which the insertion section 100 is inserted. A window for imaging is provided on the distal end surface of this distal end 101, and various units such as an imaging element and an imaging optical system are stored inside the distal end 101. The present device 1 can be incorporated inside the distal end 101 so that the light control element 8 forms a part of the imaging optical system.

Here, when the direction in which the active bending portion 102 and the passive bending portion 103 extend is referred to as a longitudinal direction L of the insertion section 100, and the direction orthogonal to this longitudinal direction L is referred to as a radial direction R of the insertion section 100, as shown in FIG. 4B, the present device 1 can be incorporated inside the distal end 101 in a manner that the upper surface of the upper substrate 3 becomes the radial direction R, and the axial direction z of the rotary shaft member 6 becomes the longitudinal direction L. In the above manner, by incorporating the present device 1 in the insertion section 100 of the endoscope, the perpendicular direction with respect to the longitudinal direction L of the insertion section 100 of the endoscope (the radial direction of the insertion section 100) can be miniaturized. In other words, the configuration of the present device 1 contributes to narrowing the diameter of the insertion section 100.

Since the tilt θ of the electromagnetic drive source 12 may be set in the range of 0<θ<180°, the tilt θ of the electromagnetic drive source 12 can be set in accordance with the installation environment of the diagnostic processor in which the present device 1 is installed, that is, for example, the medical equipment or the industrial equipment such as the endoscope which is a diagnostic device, the treatment tool, or the auxiliary tool in the medical field or the industrial field. For example, if there is a dead zone in the insertion section distal end of the endoscope where members are prohibited to be installed, the electromagnetic drive source 12 can be arranged by a tilt θ so as to circumvent the dead zone. Even if the electromagnetic drive source 12 is arranged with a tilt in the above manner, for example, the size of the radial direction R of the distal end 101 of the endoscope insertion section 100 can be reduced.

### Second Embodiment

In the following, a light control device according to a second embodiment of the present invention will be explained with reference to the drawings. Detailed explanations of the portions that are the same as FIG. 1 will be omitted.

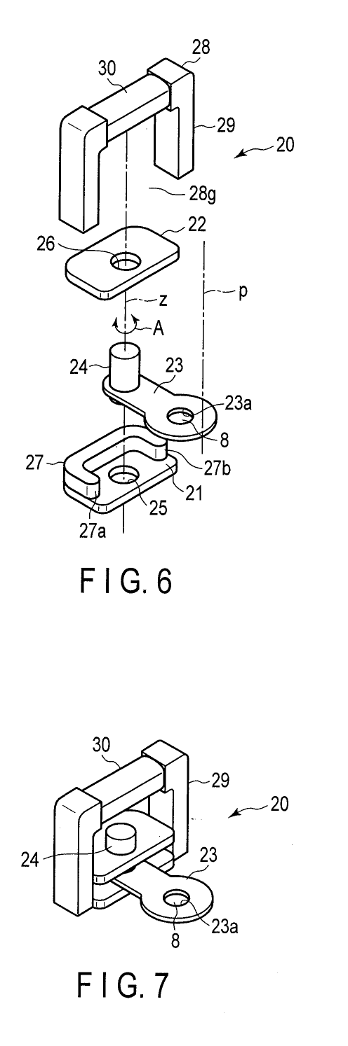

FIG. 6 shows an exploded configuration diagram of the light control device, and FIG. 7 shows an assembly diagram of the device. This light control device 20 includes a lower substrate 21 and an upper substrate 22. Each of the lower substrate 21 and the upper substrate 22 is formed in a quadrilateral.

On the lower substrate 21, for example, a shaft bearing hole 25 is arranged for bearing a rotary shaft member 24 on which a blade 23 is provided.

On the upper substrate 22, in the same manner as the lower substrate 21, for example, a shaft bearing hole 26 is arranged for bearing the rotary shaft member 24.

The lower substrate 21 and the upper substrate 22 are formed in a shape or a size that does not shield an optical path p. The lower substrate 21 and the upper substrate 22 may also be arranged at a position that does not shield the optical path p. The blade 23 is arranged with respect to the lower substrate 21 and the upper substrate 22 in a manner so that it extends from the lower substrate 21 and the upper substrate 22 as shown in FIG. 7.

In the configuration of FIG. 6, a case in which there is one optical path p is shown; however, there also may be a plurality of optical paths. The matter of a plurality of optical paths will be discussed later on.

In the same manner as the rotary shaft member 6 of the first embodiment mentioned above, the rotary shaft member 24 is formed cylindrically, and is magnetized. This rotary shaft member 24, for example, is divided in two at a plane passing through a central axis of the cylindrical rotary shaft member 24 to form two semicircular cylinders, one of which is magnetized by an N-pole, and the other of which is magnetized by an S-pole. The rotary shaft member 24 rotates inside each of the shaft bearing holes 25 and 26 in an arrow A direction with an axial direction z of the rotary shaft member 24 as the center of rotation. On the rotary shaft member 24, the blade 23 is provided in a direction perpendicular to the axial direction z of the rotary shaft member 24. On the blade 23, a hole 23a is provided to mount a light control element 8, etc. or to function as a light control element. Accordingly, since the rotary shaft member 24 is rotated to rotate the blade 23 about the rotary shaft member 24, the light control element 8 rotationally moves around the rotary shaft member 24.

A spacer 27 is provided between the lower substrate 21 and the upper substrate 22. This spacer 27 defines the interval between the lower substrate 21 and the upper substrate 22. The spacer 27 is formed in a C-shape. Both ends of the spacer 27 each serve as a stopper 27a and a stopper 27b. These stoppers 27a and 27b define a position at which the rotation of the blade 23 should be stopped when the blade 23 is rotated about the rotary shaft member 24.

The stopper 27a stops the hole 23a of the blade 23 at a first position that is away from the optical path p. The stopper 27b stops the hole 23a of the blade 23 at a second position that is on the optical path p. The stopper 27a may stop the hole 23a of the blade 23 at the second position on the optical path p, and the stopper 27b may also stop the hole 23a of the blade 23 at the first position that is away from the optical path p.

The blade 23 abuts the stopper 27a or the stopper 27b and stops at the first position or the second position. If there is an optical path at each of the first position and the second position, the hole 23a may be stopped on the optical path of one of these optical paths. In this manner, the optical path of the first or the second position can be chosen by the rotation movement of the blade 23.

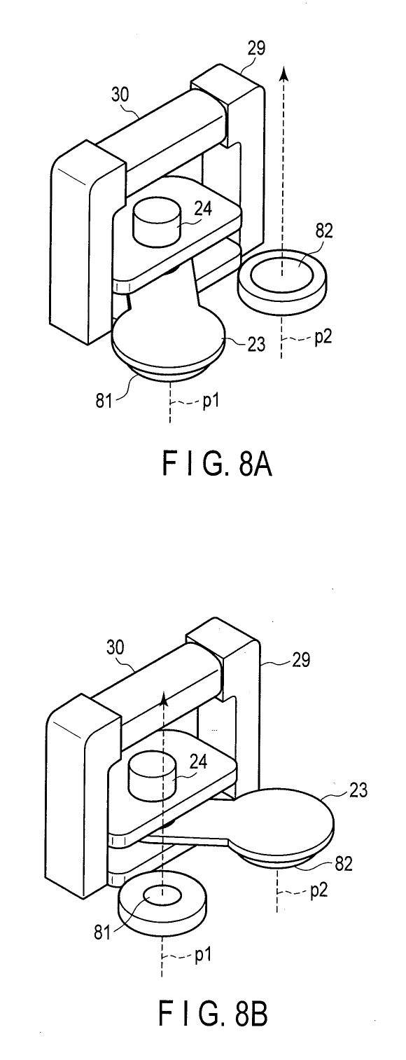

FIG. 8A and FIG. 8B show the case in dealing with such plurality of optical paths. In these drawings, the hole 23a of the blade 23 is omitted, that is, a configuration in which the light control element 8 is a shielding plate is described. Needless to say, for example, the light control element 8, such as a shutter, a lens, or a filter may also be provided on the blade 23.

FIG. 8A shows a case in which the blade 23 abuts the stopper 27a, and is stopped at the first position. A first optical path p1 passes through the first position, and a second optical path p2 passes through the second position. In this case, a first optical unit 81 comprising a light control element such as a shutter, a lens, or a filter is arranged on the first optical path p1, and the light control element 8 performs optical control for the first optical path p1, which, in this example, is shielding.

FIG. 8B shows a case in which the blade 23 abuts the stopper 27b, and is stopped at the second position. In this case, a second optical unit 82 comprising a light control element such as a shutter, a lens, or a filter is arranged on the second optical path p2, and the light control element 8 performs optical control for the second optical path p2, which, in this example, is shielding.

This may be utilized when, for example, switching between a plurality of illumination lights, such as when the first optical path p1 is an optical path of a first illumination light that is a white light, and the second optical path p2 is an optical path of a second illumination light that is a special light with a limited wavelength.

One or both of the first and the second optical units 81 and 82 may, of course, be arranged on the upper substrate 22 side instead of the lower substrate 21 side with respect to the blade 23.

Also, in the present embodiment, in the same manner as in the first embodiment, an electromagnetic drive source 28 is provided in a tilted manner against the upper surface of the upper substrate 22. This electromagnetic drive source 28 comprises, for example, a concave magnetic member 29 with a gap 28g formed thereon, and a coil 30 which is formed by being wound around this magnetic member 29. In the same manner as in FIG. 3, this coil 30 is, for example, connected to a direct-current power source 16 via a change-over switch 15.

In the above configuration, electric power is supplied to the coil 30 from the direct-current power source 16 via the change-over switch 15 to generate a magnetic field by electromagnetic induction of the coil 30. A magnetic flux of this magnetic field passes through the magnetic member 29 and the gap 28g of the magnetic member 29 to form a magnetic circuit. Since the rotary shaft member 24 is provided within the gap 28g, the magnetic field is applied to this rotary shaft member 24. Since this rotary shaft member 24 is magnetized by the N-pole and the S-pole, suction power and repulsive power are generated by the magnetic flux in the gap 28g and the N-pole and the S-pole of the rotary shaft member 24, thereby rotating the rotary shaft member 24 in the arrow A direction. The blade 23 is rotationally moved around the rotary shaft member 24 by the rotation of the rotary shaft member 24, and abuts the stopper 27a or 27b to be stopped at the first position that is away from the optical path p, or at the second position that is on the optical path p. If the optical paths p1 and p2 exist at each of the first position and the second position, the hole 23a of the blade 23 stops on one of the optical paths of these optical paths p1 and p2.

In the same manner as the electromagnetic drive source 12 in the first embodiment, the electromagnetic drive source 28 is installed in a tilted manner against the surface of the upper substrate 22 by angle θ. A tilt θ of this electromagnetic drive source 28 is set within the range of 0<θ<180°. More preferably, the tilt θ of the electromagnetic drive source 28 is set within the range of 0<θ≦90°. Furthermore, it is most favorable if the tilt θ of the electromagnetic drive source 28 is set to 90°.

According to the second embodiment configured in the above manner, since the electromagnetic drive source 28 is provided in a tilted manner against the upper surface of the upper substrate 22 by the angle θ, the same effect as the effect of the first embodiment can be exercised.

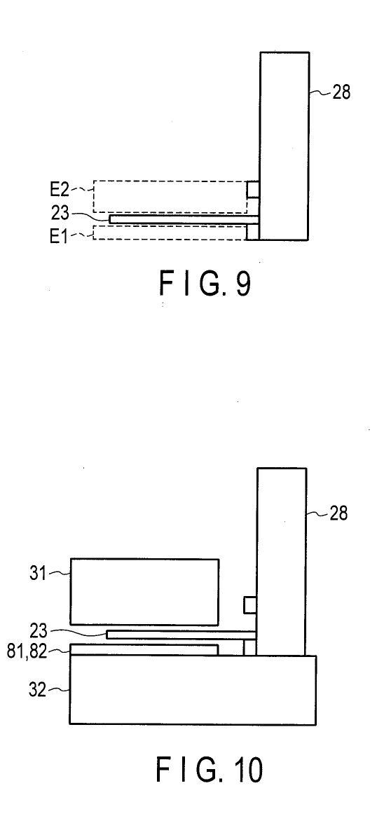

Furthermore, according to the second embodiment, since the lower substrate 21 does not exist below the moving/rotating blade 23, and the upper substrate 22 does not exist above the moving/rotating blade 23, each of spaces E1 and E2 is formed below and above the blade 23 in the manner shown, for example, in FIG. 9. In this manner, the present device 20 can be made thinner by each of the spaces E1 and E2, thus, can be made smaller. For example, by installing the present device 20 in an endoscope, the perpendicular direction with respect to the longitudinal direction L of the insertion section 100 of the endoscope (the radial direction R of the insertion section 100) can be made smaller.

As shown in FIG. 10, in the spaces E1 and E2, various units 31 and 32 such as an imaging element or an illumination light output section, etc. provided on a medical or an industrial diagnostic device, for example, the distal end 101 of the insertion section 100 of the endoscope, may be arranged. By arranging the various units 31 and 32 in the above manner, the distal end 101 of the insertion section 100 of the endoscope can be made smaller. Accordingly, in the case of rotationally moving the blade 23 in a narrow space, the present device 20 would be effective.

Therefore, by providing the present device 20 in, for example, medical equipment or industrial equipment such as an endoscope in the medical field or the industrial field, a treatment tool, or an auxiliary tool, such medical equipment and industrial equipment can be made smaller.

Since the blade 23 abuts the stopper 27a or 27b, and stops at, for example, the first position, or the second position, if the first and the second optical paths p1 and p2 are present at the first position and the second position as shown in FIG. 8A and 8B, the hole 23a of the blade 23 may be stopped by selecting one of the optical path p1 or p2 from among these optical paths p1 and p2. In this manner, for example, an optical path p1 or p2 to perform optical control by the light control element 8, such as a shutter, a lens, a shielding plate, or a filter, can be selected.

Additional advantages and modifications will readily occur to those skilled in the art. Therefore, the invention in its broader aspects is not limited to the specific details, and representative devices shown and described herein. Accordingly, various modifications may be made without departing from the spirit or scope of the general inventive concept as defined by the appended claims and their equivalents.

What is claimed is:

1. A light control device that performs, on an optical path of a light to be received by an imaging element, optical control with respect to the light, the light control device comprising:
a blade that has a distal end and a proximal end, and rotationally moves about the proximal end in a direction perpendicular to the optical path so as to be placed on and removed from the optical path;
a rotary shaft member that has one end and another end, the rotary shaft member being provided on the proximal end of the blade in a manner that perpendicularly penetrates the blade, and rotating about a rotation axis that passes through the one end and the other end, to rotationally move the blade;
a light control element that is provided on the blade, and, when positioned on the optical path by the rotational movement of the blade, performs optical control with respect to the light;
an upper substrate that is provided perpendicular to the rotary shaft member on the one end of the rotary shaft member, the upper substrate having a first upper surface, a first lower surface, and a first side surface positioned on the optical path side, and being formed to define a first space lateral to the first side surface and above the blade; and
a lower substrate that is provided perpendicular to the rotary shaft member on the other end of the rotary shaft member, the lower substrate having a second upper surface, a second lower surface, and a second side surface positioned on the optical path side, and being formed to define a second space lateral to the second side surface and below the blade.

2. The light control device according to claim 1, wherein
a first unit that causes the imaging element to receive the light is arranged in the first space, and
a second unit that causes the imaging element to receive the light is arranged in the second space.

3. The light control device according to claim 2, wherein
at least one of the first unit and the second unit is one of the imaging element and an illumination light output section.

4. A diagnostic device, comprising the light control device according to claim 1, wherein
the light control device performs optical control with respect to the light.

5. The diagnostic device according to claim 4, which is an endoscope in one of a medical field and an industrial field.
